(19) United States
(12) Patent Application Publication
(10) Pub. No.: US 2009/0076472 A1
(43) Pub. Date: Mar. 19, 2009

(54) Absorbent layer, structure and article along with a method of forming the same

(75) Inventors: Moshe D. Goldwasser, Tel Aviv (IL); Stanley R. Kellenberger, Appleton, WI (US)

Correspondence Address: WILHELM LAW SERVICE, S.C., 100 W LAWRENCE ST, THIRD FLOOR, APPLETON, WI, 54911, US

(73) Assignee: Tisteron, Ltd., Hadera (IL)

(21) Appl. No.: 11/901,636

(22) Filed: Sep. 17, 2007

(51) Int. Cl. A61F 13/15; B05D 1/36; B32B 5/16

(52) U.S. Cl. 604/365; 427/202; 428/206; 604/378; 604/384; 604/385.01; 604/385.03

(57) ABSTRACT

An absorbent layer is disclosed which is capable of absorbing a fluid and which maintains the superabsorbent particles, powder or fibers in a predetermined position. The absorbent layer is constructed of a bulky, three-dimensional fabric which has a liquid adhesive applied thereto in a non-continuous fashion. A superabsorbent is then positioned on the liquid adhesive. The absorbent layer can be secured to a liquid-impermeable outer cover to form a disposable absorbent article. A method of forming the absorbent layer is also disclosed. Furthermore, a method of forming a disposable absorbent article using the absorbent layer is described.

### FIELD OF THE INVENTION

This invention relates to an absorbent layer, an absorbent structure and an absorbent article along with a method of forming the same.

### BACKGROUND OF THE INVENTION

Various types of absorbents, products using such absorbents or absorbent layers, and various methods for forming and manufacturing absorbents and products therefrom are known today. Absorbents can be used in many different products for receiving and retaining various fluids and liquids, including but not limited to water, water based products, liquid chemicals, petroleum products including oil, crude oil, gasoline, grease, paint, etc. Absorbents are also used in disposable absorbent articles, such as infant diapers, baby diapers, training pants, sanitary napkins, catamenial pads, feminine pads, pantyliners, adult incontinent garments, bed pads, and the like. Such disposable absorbent articles are designed to be worn or utilized by humans to absorb discharged body fluids and excrement. By “disposable” it is meant articles which are designed and manufactured to be used only once and then are to be discarded after the single use. Such disposable absorbent articles can be recycled, composted or otherwise disposed of in an environmentally compatible manner. Disposable absorbent articles are not intended to be laundered, restored or otherwise reused. The typical body fluids designed to be captured by such disposable absorbent articles include urine, blood, menstrual fluid, menses, liquid feces, breast milk, sweat and perspiration. The typical excrement designed to be captured by such disposable absorbent articles include semi-solid and solid body waste, and fecal matter expelled after digestion.

It has well been recognized that thinner, disposable absorbent products are more acceptable to the consumer for they are less bulky and allow the baby, infant, toddler or child who is wearing a diaper to have greater mobility in moving his or her legs. In addition, thin disposable absorbent articles are more compact, making the product easier for the consumer to carry and store. Compactness in packaging also results in reduced distribution costs for the manufacturer and the distributor, including less shelf space. Furthermore, thin diapers are usually easier for a mother or father to secure and remove from a child and to properly dispose of in a waste receptacle. Furthermore, absorbent articles designed for older children, adults and the aged are less apparent under clothing and therefore are more discreet when worn.

One way to make a disposable absorbent article thinner while still possessing the ability to absorb and retain body fluid and body waste is to use a superabsorbent. A superabsorbent is a water-insoluble, water swellable, hydrogel polymer. The word “superabsorbent” is an abbreviation of a “superabsorbent polymer (SAP).” A superabsorbent is normally capable of absorbing large quantities of liquids such as water, body fluids, as recited above, industrial fluids or household fluids. In addition, a superabsorbent is capable of retaining such absorbed liquids under moderate pressures.

The absorption characteristics of a superabsorbent make them especially useful in designing and manufacturing disposable absorbent articles. The ability to provide thinner absorbent articles has been contingent on the ability to develop relatively thin absorbent cores or structures that can acquire and store large quantities of fluid, particularly urine. Therefore, there is a trend towards using higher concentrations of superabsorbents to achieve this purpose.

A superabsorbent is typically available in a particulate, powder or fibrous form. The superabsorbent is typically sprinkled or sifted into or onto an absorbent core formed from loosely assembled cellulose or wood pulp fluff. The absorbent core is then sandwiched between a liquid permeable cover and a liquid-impermeable outer cover. Conventional absorbent articles have the limitation that the superabsorbents are not immobilized and are free to move, migrate or shift during the manufacturing process and/or during use of the product. Movement of the superabsorbent during manufacturing can lead to absorbent material handling losses as well as to improper distribution of the superabsorbent within the finished product.

Many disposable absorbent articles include a plurality of layers, each designed for a specific purpose. For example, the upper or top layer is a liquid permeable bodyside cover which is designed to contact the body of the user and permit discharged body fluid to pass downward into the absorbent article. An acquisition/distribution layer can be located below the bodyside cover to quickly distribute the body fluid in the x, y and z directions. An absorbent core is typically located below the acquisition/distribution layer and is designed to acquire and retain the body fluid. The absorbent core is commonly constructed of wood pulp fluff and superabsorbent but may contain other fibers, such as bico bonding fiber, to assist in holding the absorbent core together and to retain the superabsorbent in place. Lastly, a liquid-impermeable outer cover is located under the absorbent core and prevents body fluid absorbed by the absorbent core from exiting the disposable absorbent article.

A number of various ways have been suggested in the prior art to keep the superabsorbent in a desired area within the disposable absorbent article. One suggestion is to use hot-melt adhesives or glue, another suggest using water swellable thermoplastic compounds. Still others suggest coating the superabsorbent particles with a resin to keep them in place. Others suggest using a filament or fiber structure, for example, a net, mesh or fibrous woven or non-woven webs. Still others suggest using an open cell foam material to maintain the superabsorbent in place. Some even suggest more esoteric ways involving using a thermoplastic component with a polar functionality to bond the superabsorbent in place. Lastly, others suggest in-situ polymerizing and/or cross-linking the superabsorbent precursor materials onto the fibers.

However, there still remains a need to arrive at an easily employable and economical way to manufacturing an absorbent layer which retains the superabsorbent in a predetermined arrangement. Now, an absorbent layer, an absorbent structure, an absorbent article and a method of forming the same have been invented which can do just that.

### SUMMARY OF THE INVENTION

Briefly, this invention relates to an absorbent layer which is capable of absorbing a fluid and which maintains the superabsorbent in a predetermined position. The absorbent layer is constructed of a three-dimensional fabric having a longitudinal central axis, a first surface, first and second ends, and first and second side edges. The fabric component of the absorbent layer has a density of less than about 0.05 g/cc. A liquid adhesive is applied to the first surface of the three-dimensional fabric in a non-continuous fashion to obtain adhesive zones and adhesive free zones. At least two of the adhesive free zones extend from the first end to the second end and each is aligned adjacent to one of the first and second side edges. A superabsorbent, in particle, powder or fibrous form, is positioned on the liquid adhesive. The superabsorbent, when in particle form, has an Absorbency Under Load (AUL) value of greater than about 13 g/g measured at 0.6 psi.

The absorbent layer can be attached to a liquid-impermeable outer cover to form a disposable absorbent article. The adhesive free zones enhance fluid flow in the absorbent layer and permit the three-dimensional fabric to be securely bonded to an adjacent layer.

A method of forming the absorbent layer is also taught. This method includes the steps of forming a three-dimensional fabric having a longitudinal central axis, a first surface, first and second ends and first and second side edges. The three-dimensional fabric also has a density of less than about 0.05 g/cc. A liquid adhesive is applied to the first surface of the three-dimensional fabric in an intermittent fashion to obtain adhesive zones and adhesive free zones. At least two of the adhesive free zones extend from the first end to the second end and each is aligned adjacent to one of the first and second side edges. A superabsorbent is then deposited onto the liquid adhesive and is compacted thereto. The three-dimensional fabric is then cut to form an individual absorbent layer.

Furthermore, a two layered absorbent structure and a disposable absorbent article are disclosed along with a method of forming each. The two layered absorbent structure includes a liquid-impermeable outer cover secured to the absorbent layer described above. The absorbent article includes three or more layers secured together. The method of forming the two layered structure includes the steps of utilizing an absorbent layer formed from a three-dimensional fabric. The three-dimensional fabric has an outer perimeter, a first surface, first and second ends, and first and second side edges. The three-dimensional fabric also has a density of less than about 0.05 g/cc. A liquid adhesive is applied to the first surface of the fabric in a non-continuous fashion to obtain adhesive zones and adhesive free zones. At least two of the adhesive free zones extend from the first end to the second end and each is aligned adjacent to one of the first and second side edges. A superabsorbent is then deposited on the liquid adhesive and is compacted thereto. A liquid-impermeable outer cover is positioned adjacent to the first surface of the absorbent layer. After being cut downstream into individual articles, the liquid-impermeable outer cover will have an outer perimeter. The absorbent layer is bonded to the liquid-impermeable outer cover by a seal which extends around at least a portion of the outer perimeters of the absorbent layer and the outer cover. The seal is located in at least some of the adhesive free zones to securely bond the absorbent layer to the outer cover.

The general object of this invention is to provide an absorbent layer, an absorbent structure and an absorbent article, each of which is capable of absorbing a fluid and which maintains the superabsorbent in a predetermined position on the absorbent layer. A more specific object of this invention is to provide an absorbent layer constructed from a three-dimensional fabric which has a liquid adhesive applied in a non-continuous fashion and which has a superabsorbent attached to the liquid adhesive.

Another object of this invention is to provide a two layered structure and a disposable absorbent article which contains the above-identified absorbent layer.

A further object of this invention is to provide a disposable absorbent article that is easy to manufacture and which maintains the superabsorbent in a predetermined arrangement.

Still another object of this invention is to provide a method of forming an absorbent layer which is capable of absorbing a fluid and which maintains the superabsorbent in a predetermined position on the absorbent layer.

Still further, an object of this invention is to provide a method of forming a two layered structure and a method of forming a disposable absorbent article which contains at least two layers and one of the layers is an absorbent layer which is capable of absorbing a fluid and which maintains the superabsorbent in a predetermined position on the absorbent layer.

Other objects and advantages of the present invention will become more apparent to those skilled in the art in view of the following description and the accompanying drawings.

### BRIEF DESCRIPTION OF THE DRAWINGS

FIG. 1 is a top view of a fabric having first and second ends and first and second side edges, and two adhesive free zones extending from the first end to the second end and each being aligned adjacent to one of the first and second side edges.

FIG. 2 is a top view of the fabric depicted in FIG. 1 and having a first surface with a liquid adhesive applied to a portion of the first surface.

FIG. 3 is a top view of the fabric depicted in FIG. 2 with a superabsorbent deposited on and adhered to the liquid adhesive to form an absorbent layer.

FIG. 4 is a top view of an alternative embodiment of the absorbent layer depicted in FIG. 3 having two additional adhesive free zones extending between the first and second side edges and each being aligned adjacent to one of the first and second ends.

FIG. 5 is a top view of still another embodiment of an absorbent layer depicted in FIG. 3 having a third adhesive free zone aligned parallel to the longitudinal central axis and being spaced apart from said first and second ends.

FIG. 6 is a top view of yet another embodiment of the absorbent layer depicted in FIG. 4 having a transverse central axis and having three additional adhesive free zones spaced apart and aligned parallel to the transverse central axis.

FIG. 7 is a top view of yet another embodiment of the absorbent layer depicted in FIG. 4 having a transverse central axis and having three additional adhesive free zones spaced apart and aligned at an angle to the transverse central axis.

FIG. 8 is a top view of still another embodiment of the absorbent layer depicted in FIG. 4 having a transverse central axis and having two additional spaced apart adhesive free zones aligned parallel to the transverse central axis and another adhesive free zone aligned parallel to the longitudinal central axis and extending from the first end to the second end.

FIG. 9 is a side view of a method of forming a disposable absorbent article.

FIG. 10 is a top view of the method depicted in FIG. 9 showing the construction of the disposable absorbent article.

FIG. 11 is a perspective view of an alternative method of forming multiple absorbent layers.

FIG. 12 is a front view of a disposable absorbent article showing a continuous seal formed inwardly of the outer periphery.

FIG. 13 is a front view of an alternative embodiment of a disposable absorbent article showing a seal formed adjacent to only the lateral side edges.

FIG. 14 is a perspective view of aligning and assembling an absorbent layer and a liquid-impermeable outer cover together.

FIG. 15 is a perspective view of four layers being assembled to form a disposable absorbent article.

FIG. 16 is a flow diagram of a method of forming an absorbent layer.

FIG. 17 is a flow diagram of a method of forming a disposable absorbent article.

FIG. 18 is a front view of a disposable waste containment article depicting a body adhesive, a seal formed inward of the outer periphery of the bodyside layer and an ingress formed through the bodyside layer through which body waste can pass from a waste orifice present in a human body.

FIG. 19 is an exploded side view of the disposable waste containment article shown in FIG. 18 taken along line 19-19, without the seal which bonds two or more of the layers together, and with a removable release layer which overlies the adhesive to prevent it from becoming contaminated prior to being attached to the skin of a human body.

### DETAILED DESCRIPTION OF THE INVENTION

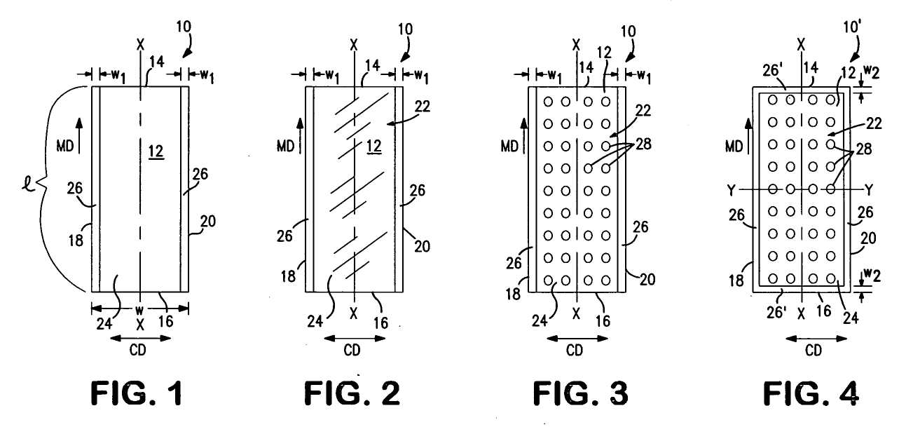

Referring to FIG. 1, a three-dimensional fabric 10 is shown having a longitudinal central axis X-X, a first surface 12, a first end 14, a second end 16, a first side edge 18 and a second side edge 20. The three-dimensional fabric 10 has a length l and a width w. The first and second ends, 14 and 16 respectively, are spaced apart and oppositely aligned relative to one another. The first and second ends, 14 and 16 respectively, are shown being aligned perpendicular to the longitudinal central axis X-X, although they could be formed at some other angle, if desired. It should also be understood that the first and second end, 14 and 16 can be non-linear, curved or arcuate in shape. Likewise the first and second side edges, 18 and 20 respectively, are spaced apart and oppositely aligned relative to one another. The first and second side edges, 18 and 20 respectively, are shown as being linear in shape and aligned parallel to the longitudinal central axis X-X, although they could have a non-linear shape and/or be tapered or be formed at an angle relative to one another. It should also be understood that the first and second side edges, 18 and 20 can be non-linear, curved or arcuate in shape.

By three-dimensional” it is meant a material having a length, a width and a thickness. Desirably, the three-dimensional fabric is bulky. By “bulky” it is meant that the three-dimensional fabric 10 has a thickness of at least about 0.5 millimeter (mm), desirably from between about 1 mm to about 5 mm, and more desirably, a thickness of from between about 1 mm to about 3 mm. The three-dimensional fabric 10 has a length measured in the machine-direction (MD), which is aligned parallel to the longitudinal central axis X-X, and a width measured in the cross-direction (CD). The cross-direction is aligned 90 degrees or at a right angle to the machine direction. The three-dimensional fabric 10 is normally processed in the machine direction. By “woven” it is meant fabrics made by interlocking fibers by means such as weaving, knitting, or the like. By “non-woven” it is meant a fabric made of one or more natural fibers and/or synthetic fibers which are held together by means other than weaving. The non-woven typically does not include woven fibers, knitted fibers, or the like. By “fabric” it is meant a structural material.

Suitable examples of processes that can be used to form a non-woven include but are not limited to: a spunbond process, a meltblown process, a coform process, a hydro-entangled process, a through air bonded carded web process, a needle punched process, and an air-laid process. Desirably, a through air bonded carded web process is utilized.

The three-dimensional fabric 10 can be formed from various materials including a renewable material. By “renewable material” it is meant a material that can be renewed, relating to or being a commodity or resource that is inexhaustible or replaceable by new growth. The three-dimensional fabric 10 can also be formed from materials that are biodegradable, biocompatible and/or compostable. By “biodegradable” it is meant a material that is capable of being decomposed by biological agents, especially bacteria. By “biocompatible” it is meant a material that is biologically compatible by not producing a toxic, injurious or immunological response in living tissue. By “compostable” it is meant a material that can be converted to compost.

Materials that work well for the three-dimensional fabric 10 are those currently used to construct acquisition layers, also commonly referred to as acquisition/distribution layers, distribution layers and/or surge layers. Such an acquisition layer is commonly employed in a disposable absorbent article, such as baby and infant diapers, child care training pants, adult incontinent products, feminine napkins, pantyliners, etc. The three-dimensional fabric 10 should allow fast penetration of body fluids, such as urine, blood, liquid feces, sweat, perspiration, etc, downward therethrough in the z-direction. In addition, the three-dimensional fabric 10 should allow the body fluid to disperse in a plane aligned parallel to the surfaces of the fabric 10 while preventing passage of superabsorbent through the fabric 10. Such a three-dimensional fabric 10 is generally characterized as being relatively open, having a low density fibrous structure with at least two different fiber sizes and a high surface area. The three-dimensional fabric 10 should have a density of less than about 0.05 grams/cubic centimeter (g/cc). Desirably, the three-dimensional fabric 10 should have a density of less than about 0.04 g/cc. More desirably, the three-dimensional fabric 10 should have a density of less than about 0.03 g/cc. Even more desirably, the three-dimensional fabric 10 should have a density of about 0.025 g/cc. The thickness of the fabric 10, for the purpose of calculating density, is determined using the combined EDANA/INDA test procedure World Supply Partners (WSP) 120.6, formerly known as “EDANA Recommended Test (ERT) 30.5.99”.

The three-dimensional fabric 10 not only takes in body fluid rapidly but should also be able to minimize the escape of the superabsorbent 28, which can be in particles, powder or fiber form. By constructing the three-dimensional fabric 10 to have sufficiently small and tortuous pores in combination with a relatively large void volume, this is accomplished. Furthermore, the fiber surface area in the bulky, three-dimensional fabric 10 (i.e. low density/large void volume) should be greater than about 1,000 cm²/g. This fiber surface area helps ensure that the escape of the superabsorbent 28 is minimized. Desirably, the fiber surface area in the three-dimensional fabric 10 is greater than about 1,500 cm²/g. More desirably, the fiber surface area in the three-dimensional fabric 10 is greater than about 2,000 cm²/g.

It should be understood that one skilled in the art can determine the surface area of fibers by the following method. For round fibers, the surface area is calculated using the formula 4/(D×ρ), where fiber diameter D is in centimeters (cm) and density ρ is in g/cc. In terms of denier d and density ρ, the formula is 3363/(d×ρ)^{1/2}.

The fiber surface area within webs composed of modified cross-section fibers (i.e. non-round fibers), such as modified cross-section staple fibers, modified cross-section melt extruded fibers, or splitable fibers can be measured by the BET method of Brunauer, Emmett and Teller, published in the Journal of the American Chemical Society, 60, 309 (1938) and discussed in many textbooks dealing with material surfaces such as the 3^{rd} addition of “Physical Chemistry of Surfaces” by Arthur W. Adamson, published by John Wiley & Sons, 1976, chapters XIII and XIV. This BET method is incorporated by reference in its entirety and made a part hereof.

The BET technique involves the absorption of a mono-molecular layer of gas molecules onto the surface of the fibers. Calculations regarding the amount of gas present on the fibers yields a quantification of the fiber surface area values. This method has been used fairly routinely in the paper industry for fibrous webs, such as papers, fillers and filter materials.

The three-dimensional fabric 10 should be somewhat compression resistant and should be relatively resilient. The three-dimensional fabric 10 can be a through air bonded, non-woven using bicomponent fibers of a first diameter joined to polyester fibers having a larger second diameter. The non-woven's bicomponent fibers can be constructed of a polyethylene core with a polypropylene sheath. A hi-loft material of this type is commercially available from Shalag Industries Ltd., Kibbutz Shamir, Upper Galilee, Israel. This material is sold as STAPTE-35 and is a hi-loft non-woven web containing polyethylene/polyester bicomponent fibers or polyethylene/polypropylene bicomponent fibers and polyester staple fibers. Other suitable materials useful as the three-dimensional fabric 10 are taught in U.S. Pat. Nos. 5,562,650 to Everett et al., 5,490,846 to Ellis et al., 5,364,382 to Latimer, et al., 5,522,810 to Allen, et al., and 5,486,166 to Bishop et al. These patents are incorporated by reference and made a part hereof.

The three-dimensional fabric 10 can also be constructed from fibers based on renewable resources (e.g. Ingeo™ fiber produced by NatureWorks, LLC, of Minneapolis, Minn.). Furthermore, the three-dimensional fabric 10 can be constructed from recycled polymers, further improving the cost effectiveness and impact on the environment. By “renewable” it is meant that which can be renewed, relating to or being a commodity or resource that is inexhaustible or replaceable by new growth. By “recycled” it is meant to extract useful materials from waste; to put or pass through a cycle again; to extract and especially reprocess materials found in waste for reuse; to use again.

Referring to FIG. 2, a liquid adhesive 22 is applied to the first surface 12. The liquid adhesive 22 can also be biodegradable, biocompatible and/or compostable. The liquid adhesive 22 can be applied in the machine-direction, in the cross-direction or at an angle to either the machine or cross-directions. The liquid adhesive 22 can be formed from various liquid based adhesives. The liquid adhesive 22 may become tacky before it solidifies. The liquid adhesive 22 can be a sprayable, synthetic elastomer based adhesive that is fast tacking and capable of low pressure spraying with minimal misting and cobwebbing. In addition, the liquid adhesive 22 should offer high coverage and a long bonding range that has low soak-in for long lasting bonds. One example of a liquid adhesive 22 which has high tack, high coverage and fast drying is polyurethane based FastBond 77, which is commercially sold by 3M Company of St. Paul, Minn. The liquid adhesive 22 can be applied by being sprayed, being applied in droplet form, by being printed, by being atomized into tiny particles or a fine spray, by being mixed with pressurized air, etc. and directed toward the three-dimensional fabric 10. By “spray” it is meant that the adhesive moves in a mass of dispersed droplets such as a fine jet of liquid discharged from a pressurized source.

Alternatively, an especially attractive printing method is one which uses a “kiss” roll, such as transfer roll printing. In this process, a “kiss” roll is partially submerged in the liquid adhesive and transfers the liquid adhesive from its surface to the fabric as the fabric passes over the rotating “kiss” roll. Methods of using this technique to apply materials in a non-continuous manner are known to those skilled in the art. In particular, the methods taught in U.S. Pat. Nos. 5,709,747; 5,885,656 and 6,183,847 teach using shields, shutters and raised areas for the non-continuous material application. These patents are incorporated by reference and made a part hereof. Other printing methods, such as gravure printing can also be used.

In general, any system of applying the liquid adhesive 22 may be utilized as long as the liquid adhesive 22 remains liquid, for example flowable or deformable, for a given period of time. A superabsorbent 28 is then positioned or deposited onto the absorbent layer 22. The adhesive should remain in a liquid or semi-liquid state, or remain tacky for a short period of time to facilitate adhering the superabsorbent 28 thereto. Furthermore, the liquid adhesive 22 should remain in a liquid or semi-liquid state, or remain tacky while the superabsorbent is compacted. More detail about applying a superabsorbent is discussed below.

The liquid adhesive 22 is applied in a non-continuous fashion to obtain at least one adhesive zone 24 and at least two adhesive free zones 26. The non-continuous fashion can be in the machine direction, the cross-direction, in both the machine and cross-directions, or at an angle to either the machine or to the cross-direction. By “non-continuous” it is meant interrupted in time, sequence, substance, or extent. The adhesive zone(s) 24 will contain the liquid adhesive 22 while the adhesive free zones 26 will not contain the liquid adhesive 22. Minor amounts of the liquid adhesive 22 may contact the adhesive free zones 26 without destroying the functionality of this invention. The amount, thickness and pattern of the liquid adhesive 22 in a given adhesive zone 24 can vary to suit one's intended needs.

Still referring to FIGS. 1 and 2, the two adhesive free zones 26, 26 are depicted as extending from the first end 14 to the second end 16. In addition, each of the two adhesive free zones 26, 26 is shown being aligned adjacent to one of the first and second side edges, 18 and 20 respectively. Each of the two adhesive free zones 26, 26 has a width w₁ which can range from between about 1 millimeter (mm) to more than 100 mm. Desirably, the width w₁ of each of the adhesive free zones 26, 26 can range from between about 2 mm to about 50 mm. More desirably, the width w₁ of each of the adhesive free zones 26, 26 can range from between about 2 mm to about 10 mm. Even more desirably, the width w₁ of each of the adhesive free zones 26, 26 can range from between about 2 mm to about 8 mm. Each of the two adhesive free zones 26, 26 can enhance fluid flow and/or facilitate bonding of the three-dimensional fabric 10 to another layer or material.

Referring now to FIG. 3, a superabsorbent 28 is positioned or deposited on the three-dimensional fabric 10. By “positioned” it is meant to place the superabsorbent 28 on the fabric 10. By “deposited” it is meant to put or set down the superabsorbent 28 on the fabric 10. The superabsorbent 28 adheres to the liquid adhesive 22 in the adhesive zone(s) 24. The superabsorbent 28 can completely cover all of the liquid adhesive 22, or alternatively, can cover only a predetermined percentage of the liquid adhesive 22. Desirably, at least about 50% of the liquid adhesive 22 is covered by the superabsorbent 28. More desirably, at least about 75% of the liquid adhesive 22 is covered by the superabsorbent 28. Even more desirably, at least about 95% of the liquid adhesive 22 is covered by the superabsorbent 28. Most desirably, 100% of the liquid adhesive 22 is covered by the superabsorbent 28.

The superabsorbent 28 is a hydrocolloidal material. Desirably, the superabsorbent 28 is formed from one or more renewable materials. The superabsorbent 28 can be a cross-linked, solution or suspension polymerized, hydrogel forming material. The superabsorbent 28 can include at least some natural based materials. Commonly, the superabsorbent 28 contains synthetics or man made materials. The superabsorbent 28 can also be biodegradable, biocompatible and/or compostable.

The superabsorbent 28 is normally added to a disposable absorbent article to increase the amount of fluid which it can acquire and also to increase its fluid retention capabilities. The superabsorbent 28 can be in the form of individual particles, in powder form or in fiber form. Desirably, the superabsorbent 28 is in particle form. The superabsorbent 28, when in particle or fiber form, should not exhibit any sharp edges or corners. For example, the superabsorbent 28, when produced with a suspension polymerization process generally does not have sharp edges or if produced with a solution polymerization process, the superabsorbent 28 can be treated or processed to remove any sharp edges or corners from the particles by methods known to those skilled in the art. This feature will help ensure that the superabsorbent 28 does not poke or form holes or openings in an adjacent layer, especially an adjacent liquid-impermeable outer cover.

The superabsorbent 28 should be generally strong, stiff and have the ability to absorb body fluid under restraining forces resulting in a superabsorbent gel bed that remains permeable when the superabsorbent 28 is swollen. Various characteristics are known to those skilled in the art for qualifying desirable superabsorbents. The superabsorbent 28, when in particle form, should have an Absorbency Under Load (AUL) value measured at 0.6 psi of greater than about 13 grams/grams (g/g). Desirably, the superabsorbent 28, when in particle form, should have an Absorbency Under Load (AUL) value measured at 0.6 psi of greater than about 20 grams/grams (g/g). More desirably, the superabsorbent 28, when in particle form, should have an Absorbency Under Load (AUL) value measured at 0.6 psi of greater than about 23 grams/grams (g/g). Even more desirably, the superabsorbent 28, when in particle form, should have an Absorbency Under Load (AUL) value measured at 0.6 psi of greater than about 25 grams/grams (g/g).

The superabsorbent 28, when in particle form, should be large enough to minimize passage of it through the three-dimensional fabric 10 and small enough to minimize discomfort and damage to any adjacent layer. Therefore, at least about 98% of the superabsorbent 28, when in particle form, should range from between about 45 micrometers to about 840 micrometers. Desirably, the particles of the superabsorbent 28 are less than about 840 micrometers. More desirably, the particles of the superabsorbent 28 range from between about 150 micrometers to about 600 micrometers. Even more desirably, the particles of the superabsorbent 28 range from between about 200 micrometers to about 600 micrometers.

The particle size of a superabsorbent material, when in particle form, may be determined by sieve size analysis. A stack of sieves with different size openings may be used to determine the particle size distribution of a given sample. For example, in principle, a particle that is retained on a sieve with 600 micrometer openings is considered to have a particle size greater than 600 micrometers.

One way to determine the superabsorbent particle size is to use sieves having 841 (U.S. Sieve No. 20), 707 (U.S. Sieve No. 25), 595 (U.S. Sieve No. 30), 210 (U.S. Sieve No. 70), 149 (U.S, Sieve No. 100), 105 (U.S. Sieve No. 140) and 44 (U.S. Sieve No. 325) micrometer openings and placing them in order of the size of the openings with the largest openings on the top of the stack and the smallest openings on the bottom of the stack. The stack is placed on the top of a pan. A 25 gram to 100 gram sample of superabsorbent particles is then placed into the sieve with the largest openings. The sieve stack is shook for 10 minutes with a Ro-Tap Mechanical Sieve Shaker, Model B, available from W. S. Tyler of Mentor, Ohio, or other similar shaking device. After shaking is complete, the superabsorbent particles retained on each sieve are weighed and recorded. The weights retained on the different size sieves are divided by the initial sample weight to determine the percent superabsorbent retained on each sieve. If the sum of the superabsorbent passing through the larger sieve and retained on the smaller sieve is greater than about 98%, the particles are considered to be within the range of interest. For example, if greater than 98% of the superabsorbent passes through the U.S. Sieve No. 20 and is retained on the U.S. Sieve No. 325, for the purpose of this invention, it has a particle size between about 45 micrometers and 840 micrometers. Similarly, if greater than 98% of the superabsorbent passes through the U.S. Sieve No. 25 and is retained on the U.S. Sieve No. 140, for the purpose of this invention, it has a particle size between about 105 micrometers and 705 micrometers.

An example of a superabsorbent 28 that has been found to be suitable for this invention is Sanwet KC-770, produced by San-Dia Polymers, Ltd, Tokyo, Japan. An example of another superabsorbent believed to be suitable for this invention is Sanwet IM-930, also produced by San-Dia Polymers, Ltd. These particular superabsorbents are partially cross linked, solution polymerized sodium polyacrylate. Other suitable superabsorbents which have a more rounded and narrower particle size distribution include Aquapearl DS50TI, also produced by San-Dia Polymers, Ltd. and Aqua Keep SA55SX II, produced by Sumitomo Seika Chemicals Company, Ltd of Osaka, Japan. These last two superabsorbents are suspension polymerized, partially cross linked, sodium polyacrylate superabsorbents. Still other acceptable superabsorbents that can be used in this invention include superabsorbents available from BASF, Charlotte, N.C.; Degussa, Greensboro, N.C.; superabsorbents from Nippon Shokubai, Osaka, Japan; and superabsorbent fibers (SAF), e.g. sold as Oasis, by Technical Absorbents Ltd., Grimsby, United Kingdom. Still further, other suitable hydrogel forming materials include those beginning with natural based resources are available from various venders. SuperNatural Absorbing polymers (SNAPs) are manufactured by Archer Daniels Midland having an office in Decatur, Ill. Biocompatible, biodegradable polymers, such as those taught in U.S. Pat. No. 6,833,488, may be used. The teachings of U.S. Pat. No. 6,833,488 are incorporated by reference and made a part hereof.

The superabsorbent 28 can be applied to the three-dimensional fabric 10 in various ways. These ways include but are not limited to: being positioned, being deposited, being dropped by gravity, being metered, being blown, being sifted, being applied using a vacuum or suction, or by other means known to those skilled in the art. The superabsorbent 28 can be homogenously applied to the three-dimensional fabric 10 to create a uniform distribution of particles, powder or fibers in the machine-direction and/or in the cross-direction. Alternatively, the superabsorbent 28 can be applied in a non-uniform pattern onto the three-dimensional fabric 10. Desirably, the superabsorbent 28 is positioned or deposited in particle form onto the three-dimensional fabric 10 as the fabric 10 is being advanced or moved in the machine direction. The superabsorbent 28 can be pulsed from a hopper using solenoid valves or other devices known to those skilled in the art. It should be understood that the superabsorbent 28 will adhere to the liquid adhesive 22 used to create one or more adhesive zones 24. The superabsorbent 28 that lands on the adhesive free zones 26 will not stick to the three-dimensional fabric 10 since little, if any, liquid adhesive 22 is present in the adhesive free zones 26. Any of the superabsorbent 28 that contacts the adhesive free zones 26 can be subsequently removed so as not to interfere with the ability of the adhesive free zones 26 to bond with an adjacent layer and/or transport fluid. The superabsorbent 28 which is positioned or deposited on or is present on the adhesive free zones 26 can be removed downstream in the process by various means, including but not limited to: using vibration, using air, using pressurized air, using a vacuum or suction, using a mechanical device, etc. More will be explained about this when describing the method.

Since the liquid adhesive 22 can be applied as a spray or as a fine mist of droplets, the adhesive zone 24 can be evenly coated with the liquid adhesive 22 and the amount of superabsorbent 28 positioned or deposited thereon will be affected by the speed at which the three-dimensional fabric 10 is moving, the rate at which the superabsorbent 28 is being positioned or deposited, the size of the superabsorbent particles, powder or fibers, the distance the exit opening of the hopper which holds the superabsorbents 28 is located away from the three-dimensional fabric 10, etc. These and other conditions affecting the application of the superabsorbent 28 will be known to those skilled in the art.

Referring to FIG. 4, an alternative embodiment of a three-dimensional fabric 10′ is shown which is similar to that shown in FIG. 3 except that two additional adhesive free zones 26′, 26′ are situated adjacent to the first and second ends, 14 and 16 respectively. The two additional adhesive free zones 26′, 26′ are aligned parallel to the transverse central axis Y-Y of the three-dimensional fabric 10. Each of the two additional adhesive free zones 26′, 26′ have a width w₂ that can be smaller, equal to or be larger in size than the width w₁ of each of the other two adhesive free zones 26, 26. Desirably, the width w₂ of each of the two adhesive free zones 26′, 26′ will be equal to or will be larger than the width w₁ of each of the first two adhesive free zones 26, 26. More desirably, the width w₂ of each of the two adhesive free zones 26′, 26′ will be approximately equal to the width w₁ of each of the first two adhesive free zones 26, 26. Each of the two additional adhesive free zones 26′, 26′ intersect with the first two adhesive free zones 26, 26 at an angle of approximately 90 degrees. Furthermore, each of the two additional adhesive free zones 26′, 26′ can have a width w₂ of from between about 2 mm to more than 100 mm. Desirably, each of the adhesive free zones 26′, 26′ has a width w₂ that can range from between about 2 mm to about 50 mm. More desirably, each of the adhesive free zones 26′, 26′ has a width w₂ that can range from between about 2 mm to about 10 mm. Even more desirably, each of the adhesive free zones 26′, 26′ has a width w₂ that can range from between about 2 mm to about 8 mm. Each of the adhesive free zones 26′, 26′ can enhance fluid flow and/or facilitate bonding the three-dimensional fabric 10′ to another layer or material.

It should be understood that the three-dimensional fabric 10 or 10′ can have any desired geometrical configuration, including but not limited to: a square, a rectangular, a triangle, be round, be oval, be elliptical, be dog-bone shape, be asymmetrical, etc. Accordingly, the adhesive free zones 26, 26, 26′ and 26′ can also have any desired geometrical shape or configuration. The three-dimensional fabric 10′ can also be biodegradable, biocompatible and/or compostable.

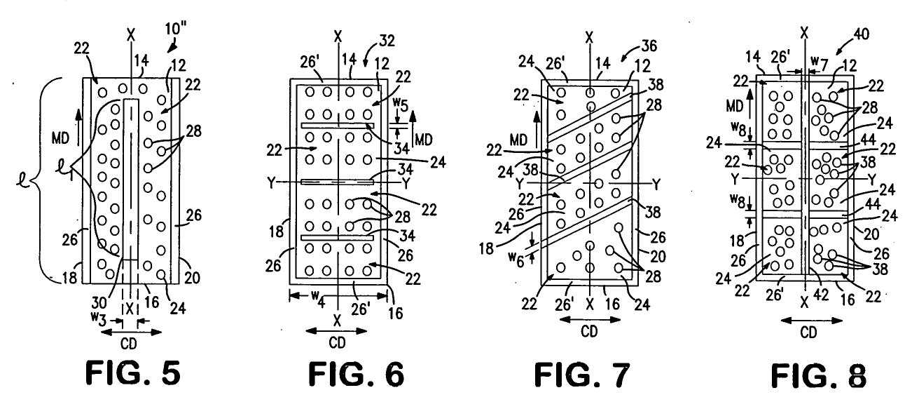

Referring to FIG. 5, another embodiment of a three-dimensional fabric 10″ is shown. The three-dimensional fabric 10″ can be woven or non-woven and can also be biodegradable, biocompatible and/or compostable. This embodiment is similar to FIG. 3 except that it includes an adhesive free zone 30. For illustration purposes only, the adhesive free zone 30 is depicted as a longitudinal channel coextensively aligned with at least a portion of the longitudinal central axis X-X. The adhesive free zone 30 has a length l₁ measured parallel to the longitudinal central axis X-X. The length l₁ extends along the length l of the three-dimensional fabric 10 but can stop short of contacting the first and second ends, 14 and 16 respectively, if desired. The length l₁ of the adhesive free zone 30 can range from between about 10% to 100% of the length l of the three-dimensional fabric 10″. Desirably, the length l₁ of the adhesive free zone 30 is at least about 50% of the length l of the three-dimensional fabric 10″. More desirably, the length l₁ of the adhesive free zone 30 is at least about 75% of the length l of the three-dimensional fabric 10″. Even more desirably, the length l₁ of the adhesive free zone 30 is at least about 85% of the length l of the three-dimensional fabric 10″. Furthermore, the adhesive free zone 30 can have a width w₃, measured perpendicular to the longitudinal central axis X-X, of from between about 1 mm to about 50 mm. Desirably, the adhesive free zone 30 can have a width w₃, measured perpendicular to the longitudinal central axis X-X, of from between about 2 mm to about 10 mm. More desirably, the adhesive free zone 30 can have a width w₃, measured perpendicular to the longitudinal central axis X-X, of from between about 2 mm to about 8 mm.

The adhesive free zone 30, along with the two adhesive free zones 26, 26, create three spaced apart adhesive free zones which are present between the first and second side edges, 18 and 20 respectively. The three adhesive free zones 26, 26 and 30 create a non-continuous pattern of liquid adhesive 22 in the cross-direction of the three-dimensional fabric 10″. It should be understood that the adhesive free zone 30 can extend from 0 degree to about 90 degrees relative to the longitudinal central axis X-X, if desired. The adhesive free zone 30 creates a non-continuous pattern of liquid adhesive 22 in the cross-direction over at least a portion of the length l of the three-dimensional fabric 10″. In short, two spaced apart adhesive zones 22 are created. The three-dimensional fabric 10″ can be cut and/or slit along the adhesive free zone 30 to create two individual absorbent layers. The adhesive free zone 30 can enhance fluid flow and/or facilitate bonding the three-dimensional fabric 10″ to another layer or material.

Referring to FIG. 6, still another embodiment of a three-dimensional fabric 32 is shown. The three-dimensional fabric 32 can be woven or non-woven and can also be biodegradable, biocompatible and/or compostable. The three-dimensional fabric 32 is similar to FIG. 4 except that it includes three additional adhesive free zones 34. The three additional adhesive free zones 34 are depicted, for illustration purposes only, as horizontal channels aligned parallel to the transverse central axis Y-Y. The three adhesive free zones 34 are spaced apart from one another and are positioned between the first and second ends, 14 and 16 respectively. Each of the three adhesive free zones 34 has a length l₂ measured parallel to the transverse central axis Y-Y. The length l₂ of each of the three adhesive free zones 34 extends along the width w₄ of the three-dimensional fabric 32 but can stop short of contacting the first and second side edges, 18 and 20 respectively, if desired. The length l₂ of each of the three adhesive free zones 34 can range from between about 10% to 100% of the width w₄ of the three-dimensional fabric 32. Desirably, the length l₂ of each of the three adhesive free zones 34 is at least 50% of the width w₄ of the three-dimensional fabric 32. More desirably, the length l₂ of each of the three adhesive free zones 34 is at least 75% of the width w₄ of the three-dimensional fabric 32. Even more desirably, the length l₂ of each of the three adhesive free zones 34 is at least 85% of the width w₄ of the three-dimensional fabric 32.

Each of the three adhesive free zones 34 has a width w₅, measured parallel to the longitudinal central axis X-X, of from between about 1 mm to about 50 mm. Desirably, each of the adhesive free zones 34 has a width w₅ ranging from between about 2 mm to about 10 mm. More desirably, each of the adhesive free zones 34 has a width w₅ ranging from between about 2 mm to about 8 mm.

Each of the three adhesive free zones 34, along with the remaining four adhesive free zones 26, 26, 26′ and 26′ create a plurality of adhesive free zones between the first and second ends, 14 and 16 respectively. The three adhesive free zones 34 create a non-continuous pattern of liquid adhesive 22 in the machine direction of the three-dimensional fabric 32. In other words, the adhesive free zones 34 are aligned perpendicular to the longitudinal central axis X-X. It should be understood that the three adhesive free zones 34 can extend from 0 degree to about 90 degrees relative to the transverse central axis Y-Y, if desired. The three adhesive free zones 34 create a non-continuous pattern of liquid adhesive 22 in the machine direction over at least a portion of the width w₄ of the three-dimensional fabric 32. In short, four spaced apart adhesive zones 22 are created. If desired, the three-dimension fabric 32 can be cut and/or slit along the adhesive free zones 34 to create four smaller size individual absorbent layers. Each of the three adhesive free zones 34 can enhance fluid flow and/or facilitate bonding the three-dimensional fabric 32 to another layer or material.

Referring now to FIG. 7, still another embodiment of a three-dimensional fabric 36 is shown. The three-dimensional fabric 36 can be woven or non-woven and can also be biodegradable, biocompatible and/or compostable. The three-dimensional fabric 36 is similar to FIG. 4 except that it includes at least three adhesive free zones 38 formed between the first and second ends, 14 and 16 respectively. The three adhesive free zones 38 are aligned at an angle to the longitudinal central axis X-X. Any desired angle can be utilized. The three adhesive free zones 38 are spaced apart from one another and each has a width w₆ which can range from between about 1 mm to about 50 mm. Desirably, each of the adhesive free zones 38 has a width w₆ ranging from between about 2 mm to about 10 mm. More desirably, each of the adhesive free zones 38 has a width w₆ ranging from between about 2 mm to about 8 mm.

The three adhesive free zones 38 create a non-continuous pattern of liquid adhesive 22 in the machine direction in the three-dimensional fabric 36. The three adhesive free zones 38 intersect with the adhesive free zones 26, 26 to create a non-continuous pattern of liquid adhesive 22 in the machine direction in the three-dimensional fabric 36. The liquid adhesive 22 can be sprayed onto the first surface 12 in a non-continuous fashion. In short, four adhesive zones 24 are created between the first and second ends, 14 and 16 respectively. As shown, three adhesive free zones 38, 38, 38 are present between the first and second side edges, 18 and 20 respectively, and four adhesive zones 24, 24, 24 and 24 are present. If desired, the three-dimension fabric 36 can be cut and/or slit along one or more of the adhesive free zones 38 to create smaller sized individual absorbent layers. Each of the adhesive free zones 38 can enhance fluid flow and/or facilitate bonding the three-dimensional fabric 36 to another layer or material.

Referring to FIG. 8, still another embodiment of a three-dimensional fabric 40 is shown. The three-dimensional fabric 40 can be woven or non-woven and can also be biodegradable, biocompatible and/or compostable. The three-dimensional fabric 40 is similar to FIG. 4 except that it includes an adhesive free zone 42 aligned along the longitudinal central axis X-X and extending parallel to the first and second side edges 18 and 20. The adhesive free zone 42 has a width w₇ which can range from between about 1 mm to about 50 mm. Desirably, the adhesive free zone 42 has a width w₇ ranging from between about 2 mm to about 10 mm. More desirably, the adhesive free zone 42 has a width w₇ ranging from between about 2 mm to about 8 mm. The adhesive free zone 42 intersects the two adhesive free zones 26′, 26′ positioned adjacent to the first and second ends, 14 and 16 respectively. Two additional adhesive free zones 44, 44 are aligned parallel to the transverse central axis Y-Y. The two adhesive free zones 44, 44 are spaced apart from one another and each has a width w₈ which can range from between about 1 mm to about 50 mm. Desirably, each of the adhesive free zones 44, 44 has a width w₈ ranging from between about 2 mm to about 10 mm. More desirably, each of the adhesive free zones 44, 44 has a width w₈ ranging from between about 2 mm to about 8 mm. Each of the two adhesive free zones 44, 44 intersect the adhesive free zone 42 to create a non-continuous pattern of liquid adhesive 22 in the machine direction and in the cross-direction of the three-dimensional fabric 40. In short, six adhesive zones 24 are created. If desired, the three-dimension fabric 40 can be cut or slit along one or more of the adhesive free zones 26′, 26′, 42, 44 and 44 to create smaller sized individual absorbent layers. If additional individual absorbent layers are is desired, one merely has to increase the number of adhesive free zones 42 and/or 44, 44. Each of the adhesive free zones 26, 26, 26′, 26′, 42, 44 and 44 can enhance fluid flow and/or facilitate bonding the three-dimensional fabric 40 to another layer or material.

### Method

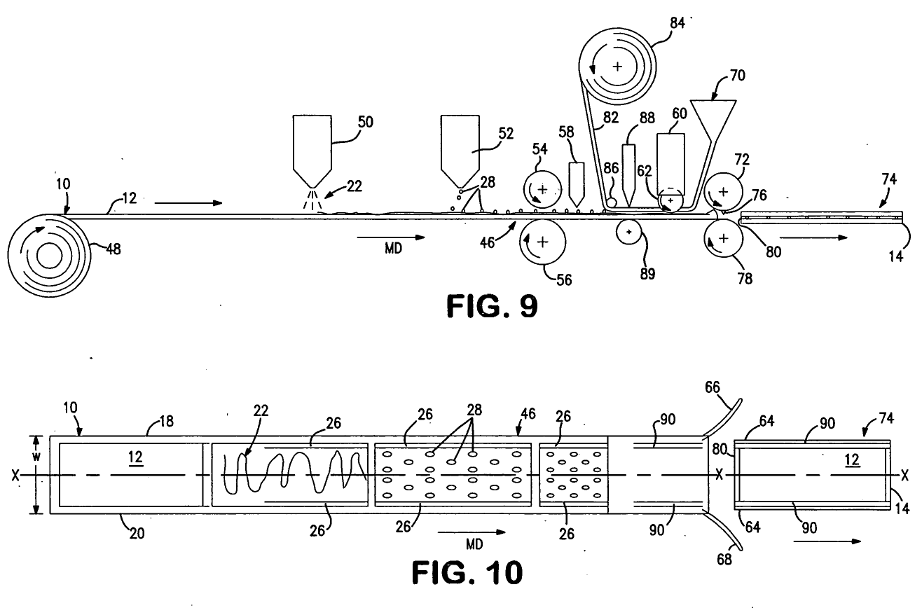

Referring now to FIGS. 9 and 10, a method of forming an absorbent layer 46 will now be explained. The method of forming the absorbent layer 46 includes the steps of utilizing a three-dimensional fabric 10, 10′, 10″, 32, 36 or 40 as described above. The three-dimensional fabric 10, 10′, 10″, 32, 36 or 40 is unwound from a supply roll 48. Alternatively, the three-dimensional fabric 10, 10′, 10″, 32, 36 or 40 can be formed in situ. The three-dimensional fabric 10, 10′, 10″, 32, 36 or 40 has a density of about 0.05 g/cc. The three-dimensional fabric 10, 10′, 10″, 32, 36 or 40 also has a longitudinal central axis X-X, which is coextensive with the longitudinal central axis X-X of the absorbent layer 46, see FIG. 10. The three-dimensional fabric 10, 10′, 10″, 32, 36 or 40 has a first surface 12 and first and second ends 14 and 16 respectively. Only the first end 14 is visible in FIG. 9 or 10 since the second end 16 is located at the center of the supply roll 48. The three-dimensional fabric 10, 10′, 10″, 32, 36 or 40 also has first and second side edges, 18 and 20 respectively.

A liquid adhesive 22 is applied to the first surface 12 from a spray nozzle 50. One or more spray nozzles 50 can be utilized. The spray nozzles 50 can be arranged across the width w or w₄ of the three-dimensional fabric 10, 10′, 10″, 32, 36 or 40 and/or along a portion of the length l of the three-dimensional fabric 10, 10′, 10″, 32, 36 or 40. The liquid adhesive 22 is shown being applied as a spray wherein droplets of adhesives dispersed in pressurized air are directed toward the first surface 12 of the three-dimensional fabric 10, 10′, 10″, 32, 36 or 40. The liquid adhesive 22 could be applied by other means known to those skilled in the art as recited above. The liquid adhesive 22 is applied in a non-continuous fashion, perpendicular to the longitudinal central axis X-X, to obtain an adhesive zone 24 and two or more adhesive free zones 26. The size and shape of the adhesive zone 24 and the adhesive free zones 26 can vary to suit one's intended uses of the absorbent layer 46. Likewise, the exact number of adhesive zones 24 and adhesive free zones 26 can also vary. The two adhesive free zones 26 extend from the first end 14 to the second end 16 and each is aligned adjacent to one of the first and second side edges, 18 and 20 respectively. Any one of the additional adhesive free zone 26′, 26′, 30, 34, 38, 42 and/or 44, is optional. If an additional adhesive free zone 26′, 26′, 30, 34, 38, 42 and/or 44 is present, each is located between the other two adhesive free zones 26, 26. Any of the additional adhesive free zone 26′, 26′, 30, 34, 38, 42 and/or 44 can be aligned parallel to the longitudinal central axis X-X or be aligned at an angle thereto. Any angle from between 0 degree to about 180 degrees can be utilized.

Alternatively, the spray nozzles 50 may be replaced with a printing method, as described earlier, wherein; for example, a “kiss” roll is utilized.

The method further includes positioning or depositing a superabsorbent 28 onto the liquid adhesive 22 from a hopper 52. The liquid adhesive 22 will be in a liquid or tacky state when the superabsorbent 28 is positioned or deposited on it. The hopper 52 should be capable of holding a large quantity of the superabsorbent 28. As mentioned above, the superabsorbent 28 can be in particle, powder or fiber form. Desirably, the superabsorbent will be in particle form. The superabsorbent 28 is then compacted into the liquid adhesive 22, before the liquid adhesive 22 completely solidifies, by a pair of nip rolls 54 and 56. Alternatively, the superabsorbent 28 could be compacted into the liquid adhesive 22 after it has partially solidified. The nip roll 54 is shown rotating in a counter clockwise direction while the nip roll 56 is rotated in a clockwise direction. Alternatively, the upper roll 54 could be a brush roll rotating in the clockwise direction while the lower roll 56 also rotates in the clockwise direction. In this scenario, the superabsorbent 28 will be compacted into the liquid adhesive 22 by the brush roll 54 while any loose superabsorbent 28 is simultaneously removed by the brush roll 54.

It should also be understood that other mechanisms capable of exerting a pressure on the superabsorbent 28 can be used to compact the superabsorbent 28 into the liquid adhesive 22. The nip formed between the nip rolls 54 and 56 can be adjusted to change the amount of pressure exerted on the superabsorbent 28. Likewise, the speed of the nip rolls 54 and 56, their diameter, the material from which they are constructed, etc. can all be varied to suit one's particular requirements. The compaction step will ensure that a sufficient quantity of the superabsorbent 28 is adhered by the liquid adhesive 22 to the first surface 12 of the three-dimensional fabric 10, 10′, 10″, 32, 36 or 40.

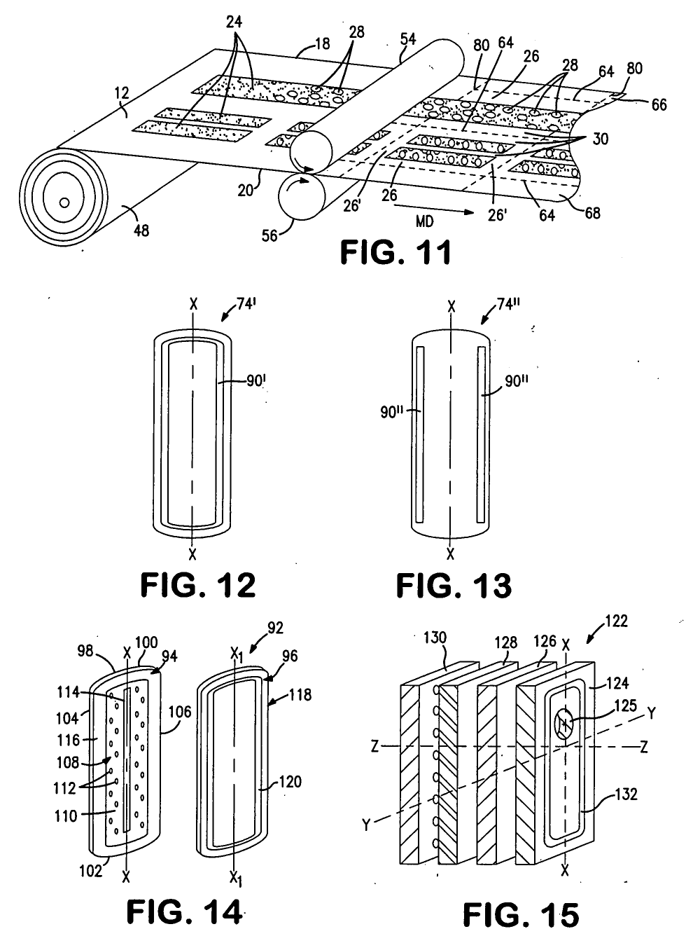

Referring to FIG. 11, an alternative arrangement for creating multiple adhesive zones 24 and multiple adhesive free zones 26, 26, 26′, 26′ and 30 is depicted. Several different size and/or shapes of adhesive zones 24 can be formed on the three dimensional fabric 10, 10′, 10″, 32, 36 or 40 by spraying or applying the liquid adhesive 22 onto the first surface 12. Each of the adhesive zones 24 can be surrounded or separated by one or more adhesive free zones 26, 26, 26′, 26′ or 30. The superabsorbent 28 is positioned or deposited onto the three dimensional fabric 10 and will adhere to the liquid adhesive 22 to form the adhesive zones 24. The superabsorbent 28 is compacted into the liquid adhesive 22 by the pair of nip rolls 54 and 56. Any excess superabsorbent 28 that has not adhered to the liquid adhesive 22 can be removed downstream of the pair of nip rolls 54 and 56.

After the compaction step, any loose superabsorbent 28 is removed. In FIG. 9, a removing device 58 is shown as a vacuum. The vacuum represents one way of removing any excess superabsorbent 28 that has not adhered to the liquid adhesive 22. It should be understood that any loose superabsorbent 28 can also be removed by other means, including but not limited to: the use of vibration, by using an inclined path over which the three-dimensional fabric 10, 10′, 10″, 32, 36 or 40 advances, by using suction, by brushing off excess superabsorbent, by using a reciprocating broom, manually, etc. It should also be recognized that if a measured amount of the superabsorbent 28 is positioned or deposited onto the liquid adhesive 22, that there may not be any excess superabsorbent 28 to remove. In this case, the removing device 58 will not be needed.

Still referring to FIGS. 9-11, the method further includes optionally using a slitter 60 having one or more rotatable trim wheels 62 to trim excess material off the first and second side edges, 18 and 20 respectively, of the three-dimensional fabric 10, 10′, 10″, 32, 36 or 40. One slitter is shown in FIG. 9, but it is to be understood that two or more slitters could be used to slit both longitudinal side edges 18 and 20 of the three-dimensional fabric 10, 10′, 10″, 32, 36 or 40 and to slit the three-dimensional fabric 10, 10′, 10″, 32, 36 or 40 in other locations. Each slitter 60 has a rotatable trim wheel 62 that can form a continuous slit line 64, see FIGS. 10 and 11. In FIG. 11, the three slit lines 64, 64, 64 are indicated as dash lines simply to show where they will occur. In FIG. 10, the first and second side edges 18 and 20 are shown being partially trimmed away from the first surface 12. The trimming does not eliminate the adhesive free zones 26, 26 but will reduce their overall width. As the longitudinal edges of the three-dimensional fabric 10, 10′, 10″, 32, 36 or 40, are longitudinally trimmed, a pair of trim sections 66 and 68 are formed, see FIGS. 10 and 11. The pair of trim sections 66 and 68 can be directed away by vacuum or by other means known to those skilled in the art to a collection device 70, see FIG. 9. The trimmed off sections 66 and 68 can be chopped up into small pieces and be recycled so as to form a new fabric. Alternatively, the trimmed off sections 66 and 68 can be used to make other materials or articles.

Still referring to FIGS. 9-11, the method further includes using a cutter 72 to cut the three-dimensional fabric 10, 10′, 10″, 32, 36 or 40 to form individual absorbent layers 46 or to form individual, two layer structures 74 which will be explained later. In FIG. 9, the cutter 72 is depicted as a rotatable cylinder having at least one knife 76 formed thereon. Multiple, spaced apart knives 76 can also be used, if desired. The knife 76 cooperates with an anvil roll 78 to assist in cleanly cutting through the three-dimensional fabric 10, 10′, 10″, 32, 36 or 40. The cut made by the knife 76 can be aligned perpendicular to or be aligned at an angle to the longitudinal central axis X-X.

It should be understood that even more complicated shapes can be cut by using, for example, rotary die cutters.

In FIG. 10, a cut line 80 is shown being formed at a right angle or at 90 degrees to the longitudinal central axis X-X. The cut line 80 could be formed at an angle to the longitudinal central axis X-X, if desired. The cut line 80 can be a linear line or a non-linear line. Examples of a non-linear line include, but are not limited to: a round line, a curve line, a partially oval line, a partially elliptical line, a semi-circular line, etc. The cut line 80 can also have any other geometrical shape or configuration, for example, a sinusoidal configuration.

In FIG. 11, two locations are depicted where the cut lines 80 will occur. As the slit lines 64, 64, 64 and the cut lines 80, 80 indicate, the three dimensional fabric 10, 10′, 10″, 32, 36 or 40 can be both slit and cut into a plurality of individual absorbent layers 46 either before a liquid-impermeable outer cover 82 is applied or after it is applied. Desirably, both the absorbent layers 46 and the liquid-impermeable outer cover 82 are slit and cut after being secured together.

Returning again to FIGS. 9 and 10, the diagrams further show a method of forming individual, two layer structures 74. Each of the two layer structures 74 includes the absorbent layer 46, as described above, in combination with a liquid-impermeable outer cover 82. The liquid-impermeable outer cover 82 or baffle is designed to prevent the passage of fluid or liquid therethrough. However, the liquid-impermeable outer cover 82 can be constructed to allow or permit the passage of air and/or moisture vapor therethrough while serving to block the passage of fluids or liquids therefrom. The liquid-impermeable outer cover 82 could be an air permeable, microporous film which will prevent fluids and liquids from passing therethrough. The liquid-impermeable outer cover 82 can be a thermoplastic film having a thickness of less than about 50 micrometers. Desirably, the liquid-impermeable outer cover 82 has a thickness of less than about 40 micrometers. More desirably, the liquid-impermeable outer cover 82 has a thickness of less than about 30 micrometers. Even more desirably, the liquid-impermeable outer cover 82 has a thickness of less than about 25 micrometers. The liquid-impermeable outer cover 82 can also be biodegradable, biocompatible and/or compostable.

Two thermoplastic films which work well for the liquid-impermeable outer cover 82 are polyethylene and polypropylene. The thermoplastic films can be tinted or made of a special color, such as blue, peach or off white, to make them more attractive.

The liquid-impermeable outer cover 82 could also be formed from a closed cell foam material. Examples of closed cell foam materials include, but are not limited to: a polyolefin foam or a polyurethane foam. A polyolefin foam can be made from polyethylene or polypropylene. One skilled in the art could also form the liquid-impermeable outer cover 82 from other materials which are capable of serving the same function.

Referring again to FIG. 9, the free end of the liquid-impermeable outer cover 82 is pulled and directed away from a supply roll 84. As the liquid-impermeable outer cover 82 is unwound from the supply roll 84, it is brought into contact with the first surface 12 of the three-dimensional fabric 10, 10′, 10″, 32, 36 or 40. One or more guide rolls 86, with only one guide roll 86 being shown, can be used to accomplish the correct orientation of the liquid-impermeable outer cover 82 to the three-dimensional fabric 10, 10′, 10″, 32, 36 or 40. The guide roll 86 allows the liquid-impermeable outer cover 82 to be aligned parallel to the first surface 12 of the three-dimensional fabric 10, 10′, 10″, 32, 36 or 40. Once the liquid-impermeable outer cover 82 is positioned on the three-dimensional fabric 10, 10′, 10″, 32, 36 or 40, it can be bonded thereto by a bonder 88. In FIG. 9, the liquid-impermeable outer cover 82 is shown being positioned vertically above the absorbent layer 46. One or more bonders 88 can be utilized, if desired.

The bonder 88 can be any type of bonder known to those skilled in the art. The bonder 88 can cooperate with an anvil roll 89, when necessary. For example, the bonder 88 could be an ultrasonic bonder which cooperates with the anvil 89. Alternatively, the bonder 88 can be a heat bonder, a pressure bonder, or a heat and pressure bonder. The bonder 88 is capable of forming one or more seals 90 which secure the liquid-impermeable outer cover 82 to at least a portion of some of the adhesive free zones 26, 26, 26′, 26′, 30, 34, 38, 42 and/or 44 formed in the absorbent layer 46. Each seal 90 can be continuous, intermittent or a combination of both. It should also be understood that the seal 90 can be aligned with at least a portion of one or more of the adhesive free zones 26′, 26′, 30, 34, 38, 42 or 44. Desirably, the seal 90 will be aligned with at least one of the adhesive free zones 26, 26, 26′, 26′, 30, 34, 38, 42 or 44. More desirably, the seal 90 will be aligned with at least a portion of one or more of the adhesive free zones 26, 26, 26′, 26′, 30, 34, 38, 42 or 44.

Referring again to FIG. 10, two parallel, spaced apart seals 90, 90 are shown which extend along the entire length of the finished disposable absorbent article 74.

Referring now to FIGS. 12 and 13, two additional embodiments are depicted of two layer structures 74′ and 74″. In FIG. 12, the two layer structure 74′ has a single continuous seal 90′ formed as an elongated oval. It should be understood that the seal 90′ could alternatively be in the shape of a circle, a rectangle, a square, an ellipse, etc. or in any other desired geometrical configuration. In FIG. 13, the two layer structure 74″ has a pair of seals 90″, 90″ each of which terminates short of the distal ends. Each of the pair of seals 90″, 90″ is a continuous linear line. Alternatively, each seal 90″ could be two or more continuous lines spaced apart from one another, be an intermittent line, be two or more intermittent lines, or be a continuous line extending over a predetermined distance and then merging into an intermittent line extending over another predetermined distance.

Referring now to FIG. 14, a two layer structure 92 is shown which includes an absorbent layer 94 and a liquid-impermeable outer cover 96. The absorbent layer 94 has an elongated oval shaped outer periphery 98 with convex ends 100 and 102, and spaced apart side edges 104 and 106. The absorbent layer 94 also has an adhesive zone 108 which includes a liquid adhesive 110 and a plurality of superabsorbent particles 112. The adhesive zone 108 completely surrounds an adhesive free zone 114. The adhesive free zone 114 is shaped as a narrow elongated channel. The adhesive zone 108 in turn is surrounded by an adhesive free zone 116. The adhesive free zone 116 extends 360 degrees around the adhesive zone 108. The liquid-impermeable outer cover 96 has an outer periphery 118 which is coterminous with the outer periphery 98 of the absorbent layer 94. This arrangement can be accomplished by cutting both the absorbent layer 94 and the liquid-impermeable outer cover 96 with a single oval shaped die or individually cutting each layer, 94 and 96, to a predetermined size and shaped configuration before the two layers 94 and 96 are aligned adjacent to one another.

The absorbent layer 94 and the liquid-impermeable outer cover 96 are aligned adjacent to each other such that the superabsorbent particles 112 face towards the liquid-impermeable outer cover 96. In use, the liquid-impermeable outer cover 96 will face away from the user's body. The liquid-impermeable outer cover 96 will contact the undergarment of the user while the non-superabsorbent surface of the absorbent layer 94 will face towards the user's skin. This results in the superabsorbent particles 112 being positioned away from the user's skin. The two layer structure 92 can be combined with one or more additional layers to form a disposable waste containment article or optionally, it could be used to absorb liquid waste, such as urine. The liquid-impermeable outer cover 96 is bonded to the absorbent layer 94 by a seal 120. The seal 120 can be formed by using ultrasonics, heat, pressure, a combination of heat and pressure, or by any other means known to those skilled in the art. The seal 120 is formed inwardly of the outer peripheries 98 and 118. The seal 120 is aligned with at least a portion of the adhesive free zone 116.

Referring to FIG. 15, a disposable absorbent article 122 is shown constructed of four layers. The disposable absorbent article 122 has a longitudinal axis X-X, a transverse axis Y-Y and a vertical axis Z-Z. The disposable absorbent article 122 includes a liquid permeable, bodyside cover 124 which is designed to contact the user's skin and has the ability to allow body fluid to pass therethrough in the Z-Z direction. The bodyside cover 124 also has an enlarged aperture 125 formed therethrough which will allow liquid, semi-solid and solid body waste to easily pass into the disposable absorbent article 122. Optionally, the bodyside cover 124 can be liquid-impermeable wherein all the body waste will pass through the enlarged aperture 125. Located adjacent to the bodyside cover 124 is an acquisition/distribution layer 126. The acquisition/distribution layer 126 is designed to allow body fluid to pass therethrough in the Z-Z direction, as well as having the ability to wick the body fluid in both the X-X and the Y-Y directions. This wicking ability facilitates better distribution of the body fluid that has insulted the disposable absorbent article 122. Located adjacent to the acquisition/distribution layer 126 and away from the bodyside cover 124 is an absorbent layer 128. The absorbent layer 128 can be constructed as shown in FIGS. 1-8. The absorbent layer 128 will function as explained above for absorbent layers 46 or 94 and has the ability to retain and hold the body fluid that has insulted the disposable absorbent article 122. Located adjacent to the absorbent layer 128 and away from the acquisition/distribution layer 126 is a liquid-impermeable outer cover 130. The liquid-impermeable outer cover 130 serves the same function as the outer covers 82 and 96 explained above. The liquid-impermeable outer cover 130 is located farther away from the user's skin but in direct contact with the user's undergarments or outer clothing. If the two layered structure 74, 74′, 74″ or 92 is used, a separate outer cover 130 would not be needed.

The liquid-impermeable outer cover 130 is capable of preventing any body fluid which has entered the disposable absorbent article 122 from being able to escape and possibly contacting and soiling the user's undergarment or outer clothing. All of the layers 124, 126, 128 and 130 are bonded together by a seal 132 which is located inward of the outer peripheries of the various layers. The seal 132 is depicted as having a racetrack configuration, although any other profile could also be used. All of the layers 124, 126, 128 and 130 of the disposable absorbent article 122 can be also be biodegradable, biocompatible and/or compostable.

It should be understood that a disposable absorbent article can be constructed out of two or more layers. The absorbent layer 10, 10′, 10″, 32, 36, 40, 94, or 128 can be bonded to a liquid-impermeable outer cover 82, 96 or 130. A liquid permeable bodyside cover 124 can be added to form a three layer structure. Likewise, an acquisition/distribution layer 126 can be further added to the three layered structure to form a four layer structure. One or more additional layers, for example, a second absorbent layer, can also be added to construct a multilayered, disposable absorbent article. It should further be understood that one or more of the layers can be bonded or secured together to form an integral, disposable absorbent article. It is not necessary that all of the interior layers be bonded together in order for the disposable absorbent article to perform properly. For example, a narrow and short interior layer could be utilized that is not bonded to any layer but which is held in position by a seal formed about the outer periphery of the absorbent article that completely surrounds the narrow, short interior layer.

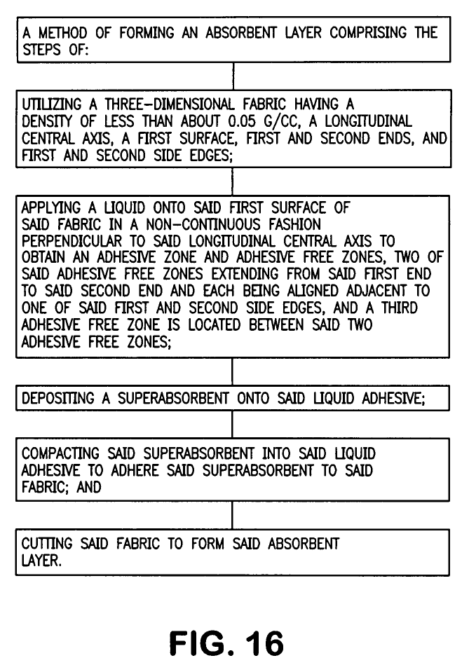

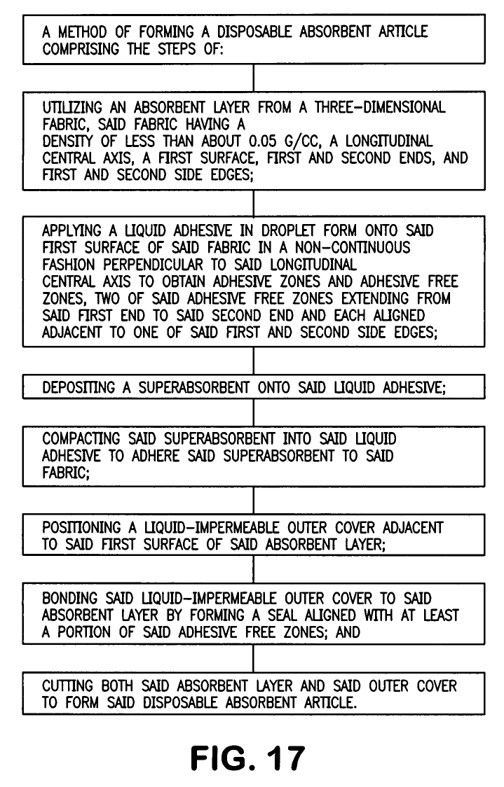

Referring to FIGS. 16 and 17, two flow diagrams are shown which depict a method of forming an absorbent layer 46 or 94, and a method of forming a disposable absorbent article 74, 74′ 74″, 92 or 122. Each flow diagram recites the steps taught above.

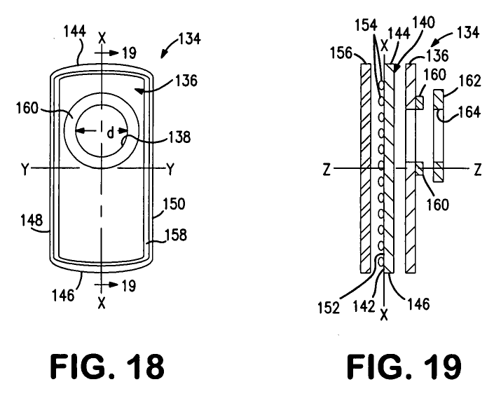

Referring to FIGS. 18 and 19, a disposable absorbent article 134 is shown. The disposable absorbent article 134 includes a bodyside cover 136 having an enlarged aperture 138 formed therethrough. The enlarged aperture 138 is depicted as a round or circular opening having a diameter d. However, the enlarged aperture 138 can have any desired geometrical shape. The diameter d, or the equivalent circular diameter (ECD) for a non-circular enlarged aperture, can vary in size in order to surround an anal opening or a urogenital area of a human body. By “ECD” it is meant the diameter of a circle having the same area as the open area of a non-circular enlarged aperture. The diameter d or the ECD should range from between about 10 millimeters (mm) to about 100 mm. Desirably, the diameter d or ECD ranges from between about 20 mm to about 75 mm. More desirably, the diameter d or the ECD ranges from between about 25 mm to about 60 mm.

The disposable absorbent article 134 also includes an absorbent layer 140 formed from a three-dimensional fabric having a density of less than about 0.05 g/cc. The absorbent layer 140 also has a longitudinal central axis X-X, a first surface 142, first and second ends, 144 and 146 respectively, and first and second side edges, 148 and 150 respectively. A liquid adhesive 152 is applied onto the first surface 142 in a non-continuous fashion and perpendicular to the longitudinal central axis X-X to obtain adhesive zones and adhesive free zones, as discussed above with reference to FIGS. 5-8 but not shown in FIGS. 18 and 19. Desirably, two of the adhesive free zones extend from the first end 144 to the second end 146 and each is aligned adjacent to one of the first and second side edges, 148 and 150 respectively. A superabsorbent 154 is secured to the liquid adhesive 152. The disposable absorbent article 134 further includes a liquid-impermeable outer cover 156 positioned adjacent to the first surface 142 of the absorbent layer 140. A seal 158 is aligned with at least a portion of the adhesive free zones and functions to secure the bodyside cover 136, the absorbent layer 140 and the outer cover 156 together to form the disposable absorbent article 134.

Lastly, referring to FIG. 19, the disposable absorbent article 134 further includes a body adhesive 160 which at least partially surround and is positioned adjacent to the enlarged aperture 138. The body adhesive 160 is capable of securing the disposable absorbent article 134 to the skin of a human body such that the enlarged aperture 138 is aligned with and surrounds a body waste orifice present in the human body. The body waste orifice can be a urethra or a urogenital area that includes the vagina in a female, the end of the penis in a male, or an anal orifice such as the anus. Desirably, the body adhesive 160 completely encircles or extends about the periphery of the enlarged aperture 138. A release layer 162 is shown covering the body adhesive 160 to prevent premature contamination thereof. The release layer 162 is depicted as optionally having an enlarged aperture 164 formed therein which is sized and configured to closely resemble the enlarged aperture 138 formed in the bodyside cover 136.

While the invention has been described in conjunction with several specific embodiments, it is to be understood that many other alternatives, modifications and variations will be apparent to those skilled in the art in light of the foregoing description. Accordingly, this invention is intended to embrace all such alternatives, modifications and variations which fall within the spirit and scope of the appended claims.

We claim:

1. An absorbent layer capable of absorbing a fluid, comprising:
a) a three-dimensional fabric having a density of less than about 0.05 g/cc, a first surface, first and second ends and first and second side edges;
b) a liquid adhesive applied to said first surface of said fabric in a non-continuous fashion to obtain an adhesive zone and adhesive free zones, at least two of said adhesive free zones extending from said first end to said second end and each being aligned adjacent to one of said first and second side edges; and
c) a superabsorbent in particle form having an Absorbency Under Load (AUL) value of greater than about 13 g/g measured at 0.6 psi, and said superabsorbent is positioned on said liquid adhesive.

2. The absorbent layer of claim 1 wherein said fabric has a machine-direction and a cross-direction, said liquid adhesive is applied in a non-continuous fashion in said cross-direction on said first surface of said fabric, and at least three adhesive free zones are present between said first and second side edges.

3. The absorbent layer of claim 1 wherein said fabric has a machine-direction and a cross-direction, said liquid adhesive is applied in a non-continuous fashion in said machine-direction on said first surface of said fabric, and at least three adhesive free zones are spaced apart between said first and second ends.

4. The absorbent layer of claim 1 wherein said absorbent layer has a longitudinal central axis and at least one adhesive free zone extends from 0 degree to about 90 degrees relative to said longitudinal central axis.

5. The absorbent layer of claim 4 wherein said adhesive free zones are aligned perpendicular to said longitudinal central axis and each adhesive free zone has a width of at least about 2 millimeters.

6. The absorbent layer of claim 1 wherein at least one of said adhesive free zones have a width of from between about 2 millimeters to about 8 millimeters and enhances fluid flow in said absorbent layer and at least one other adhesive free zone facilitates securely bonding said absorbent layer to an adjacent layer.

7. The absorbent layer of claim 1 wherein said fabric has a density of less than about 0.04 g/cc and said superabsorbent has an Absorbency Under Load (AUL) value measured at 0.6 psi of greater than about 20 g/g.

8. The absorbent layer of claim 1 wherein said superabsorbent is a partially cross-linked, polymerized, hydrogel forming material in particle form and said particles are less than about 840 micrometers and are void of any sharp edges.

9. The absorbent layer of claim 1 wherein said superabsorbent is a partially cross-linked, polymerized, hydrogel forming material in fiber form.

10. The disposable absorbent layer of claim 1 wherein at least a portion of said superabsorbent is formed from a renewable material.

11. The disposable absorbent layer of claim 1 wherein at least a portion of said fabric is formed from a renewable material.

12. A two layered structure, comprising:
a) an absorbent layer capable of absorbing a fluid, said absorbent layer including a three-dimensional fabric having a density of less than about 0.03 g/cc and having a first surface, first and second ends and first and second side edges, a liquid adhesive applied to said first surface of said fabric in a non-continuous fashion to obtain adhesive zones and adhesive free zones, at least two of said adhesive free zones extending from said first end to said second end and each being aligned adjacent to one of said first and second side edges, each of said adhesive free zones having a width of at least about 2 millimeters, and a superabsorbent is positioned on said liquid adhesive; and
b) a liquid-impermeable outer cover positioned adjacent to said first surface of said fabric and being bonded to at least one of said adhesive free zones.

13. The two layered structure of claim 12 wherein said liquid-impermeable outer cover and said fabric both have an outer perimeter, said adhesive free zones extend around said outer perimeter of said fabric, and at least a portion of said outer perimeter of said fabric is bonded to said outer perimeter of said liquid-impermeable outer cover.

14. The two layered structure of claim 13 wherein said fabric is bonded to said liquid-impermeable outer cover around said complete perimeter.

15. The two layered structure of claim 12 wherein said fabric has a second surface aligned opposite to said first surface and an acquisition/distribution layer is positioned adjacent to said second surface, a liquid permeable bodyside cover is positioned adjacent to said acquisition/distribution layer away from said fabric, and said liquid-impermeable outer cover, said fabric, said acquisition/distribution layer and said liquid permeable bodyside cover are all bonded together in at least some of said adhesive free zones.

16. The two layered structure of claim 12 wherein said fabric has a longitudinal central axis and a length measured between said first and second ends, and an additional adhesive free zone is present which extends at least about 50% of the length of said fabric and parallel to said longitudinal central axis to enhance fluid flow in said disposable absorbent article.

17. A method of forming an absorbent layer comprising the steps of:
a) utilizing a three-dimensional fabric having a density of less than about 0.05 g/cc, a longitudinal central axis, a first surface, first and second ends, and first and second side edges;
b) applying a liquid adhesive onto said first surface of said fabric in a non-continuous fashion perpendicular to said longitudinal central axis to obtain an adhesive zone and adhesive free zones, two of said adhesive free zones extending from said first end to said second end and each being aligned adjacent to one of said first and second side edges, and a third adhesive free zone is located between said two adhesive free zones;
c) depositing a superabsorbent onto said liquid adhesive;
d) compacting said superabsorbent into said liquid adhesive to adhere said superabsorbent to said fabric; and
e) cutting said fabric to form said absorbent layer.

18. The method of claim 17 further comprising removing superabsorbent that has not adhered to said liquid adhesive before said fabric is cut.

19. The method of claim 17 wherein said three-dimensional fabric has a transverse central axis and further comprising forming additional adhesive free zones in said fabric which extend parallel to said transverse central axis, said adhesive free zones permitting said fabric to be securely bonded to an adjacent layer.

20. The method of claim 17 wherein said absorbent layer has a first end and a second end, and said third adhesive free zone is spaced apart from said first and second ends of said absorbent layer, and said third adhesive free zone has a width of at least 2 millimeters to enhance fluid flow in said absorbent layer.

21. A method of forming a two layered structure comprising the steps of:
a) utilizing an absorbent layer formed from a three-dimensional fabric, said fabric having a density of less than about 0.05 g/cc, a longitudinal central axis, a first surface, first and second ends, and first and second side edges;
b) applying a liquid adhesive onto said first surface of said fabric in a non-continuous fashion and perpendicular to said longitudinal central axis to obtain adhesive zones and adhesive free zones, two of said adhesive free zones extending from said first end to said second end and each being aligned adjacent to one of said first and second side edges;
c) depositing a superabsorbent onto said liquid adhesive;
d) compacting said superabsorbent into said liquid adhesive to adhere said superabsorbent to said fabric;
e) positioning a liquid-impermeable outer cover adjacent to said first surface of said absorbent layer;
f) bonding said liquid-impermeable outer cover to said absorbent layer by forming a seal aligned with at least a portion of said adhesive free zones; and
g) cutting both said absorbent layer and said outer cover to form said disposable absorbent article.

22. The method of claim 21 wherein said two layered structure has an outer periphery and said seal is ultrasonically formed inwardly of said outer periphery and aligned with at least one of said adhesive free zones.

23. The method of claim 21 further comprising forming two additional, spaced apart adhesive free zones which extend from said first side edge to said second side edge, and which intersect with said two adhesive free zones which are aligned adjacent to one of said first and second side edges.

24. The method of claim 21 wherein a bodyside outer cover is aligned adjacent to said absorbent layer and away from said liquid-impermeable outer cover, and said bodyside outer cover, said absorbent layer and said liquid-impermeable outer cover are all bonded together in at least one of said adhesive free zones to form a disposable absorbent article.

25. A disposable absorbent article comprising:
a) a bodyside cover having an enlarged aperture formed therethrough;
b) an absorbent layer formed from a three-dimensional fabric, said fabric having a density of less than about 0.05 g/cc, a longitudinal central axis, a first surface, first and second ends, and first and second side edges, a liquid adhesive applied onto said first surface in a non-continuous fashion and perpendicular to said longitudinal central axis to obtain adhesive zones and adhesive free zones, two of said adhesive free zones extending from said first end to said second end and each being aligned adjacent to one of said first and second side edges, and a superabsorbent secured to said liquid adhesive;
c) a liquid-impermeable outer cover positioned adjacent to said first surface of said absorbent layer; and
d) a seal aligned with at least a portion of said adhesive free zones which secures said bodyside cover, said absorbent layer and said outer cover together to form said disposable absorbent article.

26. The disposable absorbent article of claim 25 wherein a body adhesive is positioned around at least a portion of said enlarged aperture, said body adhesive capable of securing said disposable absorbent article to a human body such that said enlarged aperture is aligned with and surrounds a body waste orifice present in a human body.

27. The disposable absorbent article of claim 26 further comprising a release layer covering said body adhesive to prevent premature contamination thereof.

28. The disposable absorbent article of claim 25 wherein said body waste orifice is a urethra.

29. The disposable absorbent article of claim 25 wherein said body waste orifice is an anus.
